(19) United States
(12) Patent Application Publication
(10) Pub. No.: US 2013/0289462 A1
(43) Pub. Date: Oct. 31, 2013

(54) CORRECTIONAL BRACE FOR SHOULDER REMEDIATION

(71) Applicant: Christopher Doumas, Rumson, NJ (US)

(72) Inventor: Christopher Doumas, Rumson, NJ (US)

(21) Appl. No.: 13/457,461

(22) Filed: Apr. 26, 2012

(51) Int. Cl. A61F 5/00

(52) U.S. Cl. 602/20

(57) ABSTRACT

Embodiments of the present invention relate to a correctional brace for shoulder remediation for wearing on an individual's wrist to lock the wrist in place while the individual is sleeping or resting. In one embodiment of the present invention, a correctional brace for shoulder remediation comprises a wrist guard for securely fitting around an individual's hand, the wrist guard having an anchor support on a top outer surface thereof; an anchor brace for fitting around an anchor base, the anchor brace having an anchor connection means on an outer surface thereof; and an anchor strap for anchoring the wrist guard in a substantially fixed palm anterior position, the strap connecting to the anchor support and the anchor connection means, wherein the correctional brace is capable of decompressing the individual's shoulder and subacromial space therein.

### BACKGROUND OF THE INVENTION

1. Field of the Invention

Embodiments of the present invention generally relate to a correctional brace for shoulder remediation and methods thereof. More specifically, embodiments of the present invention relate to a correctional brace for shoulder remediation for wearing on an individual's wrist to lock the wrist in place while the individual is sleeping or resting.

2. Description of the Related Art

One of the most common physical complaints heard by physicians is shoulder pain. The shoulder is made up of several joints combined with tendons and muscles that allow a great range of motion of a person's arm. Because so many different structures make up the shoulder, it is vulnerable to many different problems, including rotator cuff pathology, impingement syndrome, subacromial bursitis and arthritis.

Within the shoulder structure, impingement upon the supraspinatus tendon between the head of the humerus and the coracoacromial arch is thought to be a possible cause of rotator cuff tendonitis, subacromial bursitis and impingement syndrome. If untreated, it may lead to degeneration and further disease of the supraspinatus tendon and subsequently the rotator cuff complex. Further problems may include rotator cuff tendon tears, impingement syndrome, subacromial bursitis, and the like. Such problems may be exacerbated by flexing and internally rotating the humerus which increases the pressure on the contents of the subacromial space.

Reduction of movement which causes impingement on the supraspinatus tendon may generally be assumed to reduce its degeneration. Impingement becomes less of a potential problem when the body is upright, because the inherent weight of an individual's arm provides necessary and constant force to keep the head of the humerus unimpinged. When in a supine, or prone, position, an individual's shoulder loses the built-in traction protection provided by gravity. Maximum impingement occurs when a person's arm is in abduction, or away from the body, or in internal rotation which often happens when an individual is sleeping or resting.

As such, a device and method to limit arm abduction, internal rotation, and/or shoulder impingement, particularly directed to sleeping or resting positions, or when the body is otherwise in a supine or prone position, is of great value. Several devices have been attempted which hold the shoulder, arm or even wrist close to the body when sleeping or resting. Many of such devices function through complete restriction of movement of the arm or shoulder, which may cause frozen shoulder or adhesive capsulitis. Other devices require the arm stay close to the body, in an effort to reduce the abduction, but allow rotation. However, by permitting substantially free rotation of the arm, the overall beneficial effect is greatly lessened as the rotational movement can also lead impingement, specifically, internal rotation may cause increased pressure in the subacromial space.

Thus, there is a need for an improved correctional brace for shoulder impingement and methods thereof that greatly reduces abduction of the arm, allows the shoulder to remain unrestrained, but restricts substantial rotation of the arm. Moreover, there is a need for a correctional brace to provide gentle traction to the humerus to decompress the subacromial space.

### SUMMARY OF THE INVENTION

Embodiments of the present invention generally relate to a correctional brace for shoulder remediation and methods thereof. More specifically, embodiments of the present invention relate to a correctional brace for shoulder remediation for wearing on an individual's wrist to lock the wrist in place while the individual is sleeping or resting.

Embodiments of the present invention relate to a correctional brace for shoulder remediation comprising a wrist guard for securely fitting around an individual's hand, the wrist guard having an anchor support on a top outer surface thereof; and an anchor strap for anchoring the wrist guard in a substantially fixed palm anterior position, the strap being connected to the anchor support, and passing about an anchor base, wherein the correctional brace is capable of decompressing the individual's shoulder and subacromial space therein.

In another embodiment of the present invention, a correctional brace for shoulder remediation comprises a wrist guard for securely fitting around an individual's hand, the wrist guard having an anchor support on a top outer surface thereof; an anchor brace for fitting around an anchor base, the anchor brace having an anchor connection means on an outer surface thereof; and an anchor strap for anchoring the wrist guard in a substantially fixed palm anterior position, the strap connecting to the anchor support and the anchor connection means, wherein the correctional brace is capable of decompressing the individual's shoulder and subacromial space therein.

In yet another embodiment, a method for remediating an individual's shoulder impingement comprises providing a correctional brace comprising: a wrist guard for securely fitting around an individual's hand, the wrist guard having an anchor support on a top outer surface thereof; an anchor brace for fitting around an anchor base, the anchor brace having an anchor connection means on an outer surface thereof; and an anchor strap for anchoring the wrist guard in a substantially fixed palm anterior position, the strap connecting to the anchor support and the anchor connection means; affixing the wrist guard about the individual's wrist, and securing the wrist guard thereon; affixing the anchor brace about an anchor base and securing the anchor brace thereon; and connecting the anchor strap to the anchor support and the anchor connection means; wherein the correctional brace is capable of decompressing the individual's shoulder and subacromial space therein.

### BRIEF DESCRIPTION OF THE DRAWINGS

So the manner in which the above recited features of the present invention can be understood in detail, a more particular description of embodiments of the present invention, briefly summarized above, may be had by reference to embodiments, which are illustrated in the appended drawings. It is to be noted, however, the appended drawings illustrate only typical embodiments of embodiments encompassed within the scope of the present invention, and, therefore, are not to be considered limiting, for the present invention may admit to other equally effective embodiments, wherein:

FIG. 1 depicts a top view of a correctional brace for shoulder remediation in accordance with one embodiment of the present invention;

FIG. 2 depicts a top view of a correctional brace for shoulder remediation as worn by an individual in accordance with one embodiment of the present invention;

FIG. 3 depicts a perspective view of correctional brace for shoulder remediation having an anchor brace in accordance with one embodiment of the present invention; and

FIG. 4 depicts a flowchart of a method of utilizing a correctional brace for shoulder remediation in accordance with one embodiment of the present invention.

The headings used herein are for organizational purposes only and are not meant to be used to limit the scope of the description or the claims. As used throughout this application, the word may is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

### DETAILED DESCRIPTION

Embodiments of the present invention generally relate to a correctional brace for shoulder remediation and methods thereof. More specifically, embodiments of the present invention relate to a correctional brace for shoulder remediation for wearing on an individual's wrist to lock the wrist in place while the individual is sleeping or resting.

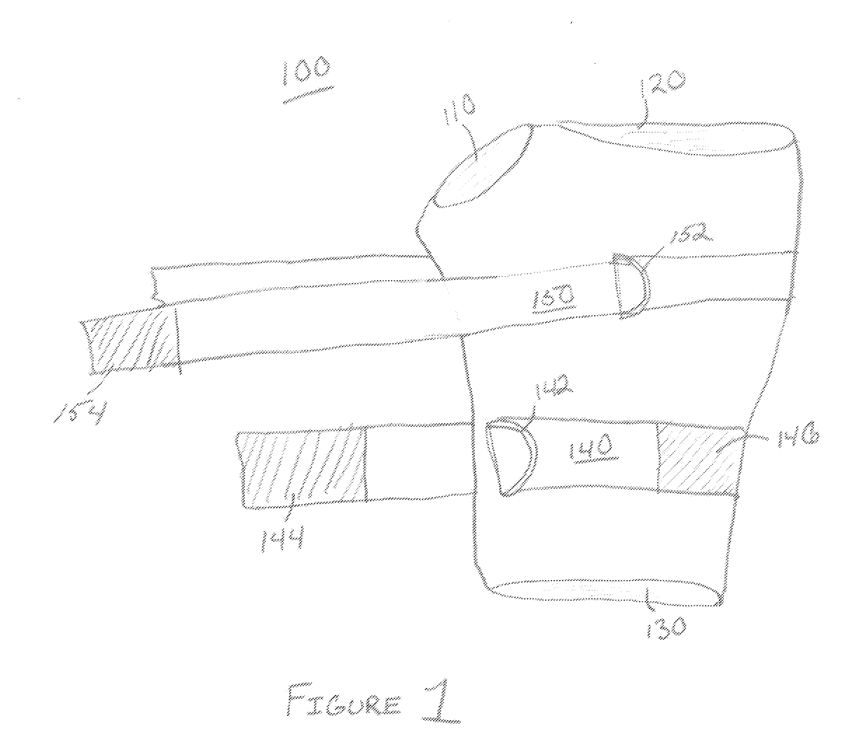

FIG. 1 depicts a top view of a correctional brace for shoulder remediation in accordance with one embodiment of the present invention. As shown in the Figure, the correctional brace 100 generally comprises a wrist guard for fitting over a user's hand via the primary opening 130, and having a thumb hole 110 and finger holes 120 for comfort.

The correctional brace 100 may generally comprise a strap 140 for retaining the wrist guard around the user's hand. The strap 140 may work in conjunction with a D-ring assembly 142, and may have fasteners, such as hook and loop fasteners thereon for ensuring a secure fit. In alternative embodiments, the strap may comprise any other type of suitable mechanical fastener, for example, snaps, buckles, clips, or the like, for holding the wrist guard in place.

In many embodiments, the correction brace 100 also comprises an anchor strap 150 and an anchor support 152 thereon. The anchor support 152 generally comprises an end of a mechanical fastener apparatus, and is generally positioned on a top surface (i.e., on the top of the user's hand, opposite the palm) of the wrist guard. The anchor support 152 may comprise any type of mechanical structure suitable for embodiments of the present invention. In one embodiment, the anchor support 152 comprises a D-ring assembly sewn into or stitched onto the top surface of the wrist guard.

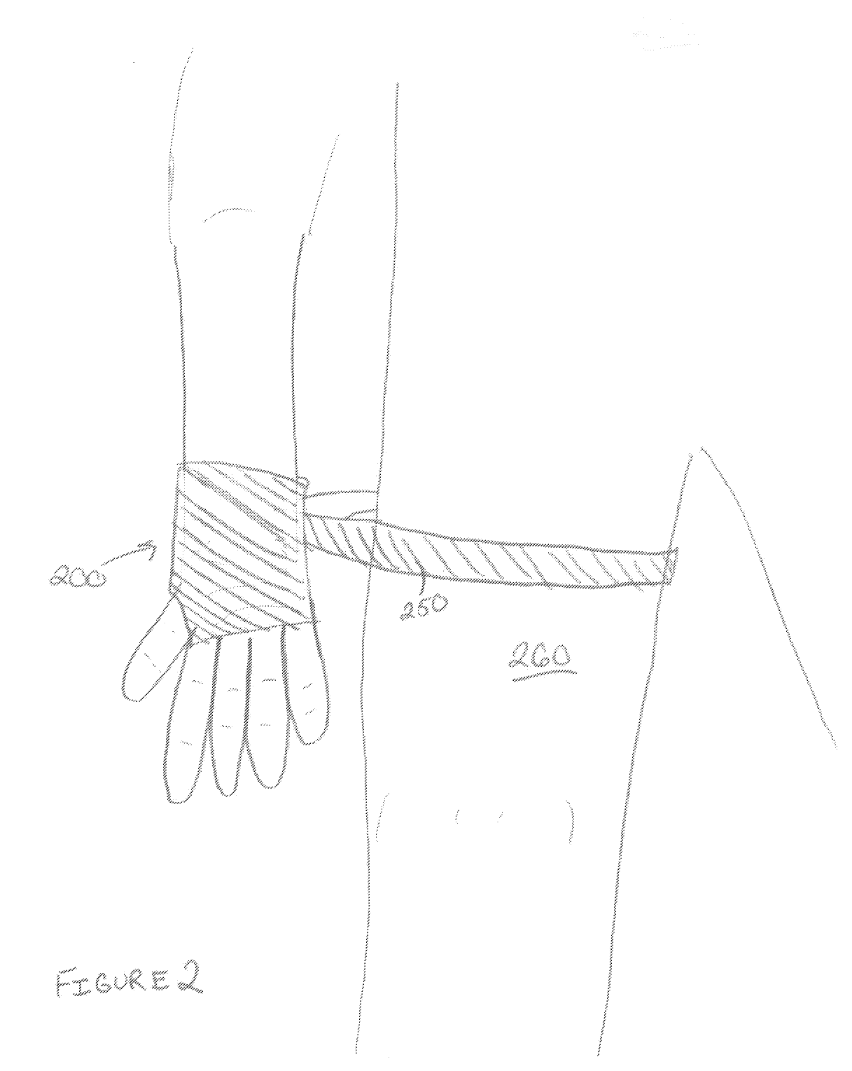

The anchor strap 150 generally serves to connect to and/or through the anchor support 152, and retain the wrist guard in a palm anterior position (i.e., palm up, or turned outward) when in use. In one embodiment, the anchor strap 150 comprises mating fasteners, such as hook and loop fasteners 154, at opposing ends, although any known fasteners may be suitable. Generally, the anchor strap 150 is sufficient long to pass around the anchor support, as well as around an anchor base, such as the individual's thigh, an article of clothing, a bed post, or the like, as shown in FIG. 2.

FIG. 2 depicts a top palm anterior view of a correctional brace for shoulder remediation as worn by an individual in accordance with one embodiment of the present invention. As shown in the Figure, the correction brace 200 may generally have the wrist guard being worn by the user in a palm anterior, i.e., palm up position, while the anchor strap 250 is positioned about an anchor base, i.e., individual's thigh 260.

In such a position, where substantially little or no space is provided between the wrist guard and the user's thigh (i.e., the anchor strap is fairly tight), the user may not be able to rotate his or her wrist inward, i.e., towards the thigh. Whereas inward rotation of the wrist would require the top of the wrist guard to rotate outward, the tension of the anchor strap should generally be such that rotation is not possible. As such, by holding the wrist in the top-down, palm up position, the shoulder is held in an optimal position for remediation and at a low risk of further impingement.

In further embodiments, the anchor strap may comprise elasticity, and it may be positioned about the anchor base, e.g., the individual's thigh, such that the strap provides constant tensile force on the individual's wrist. The traction caused by such tensile force may further assist the correctional brace 200 in decompressing the subacromial space and alleviating impingement of the shoulder.

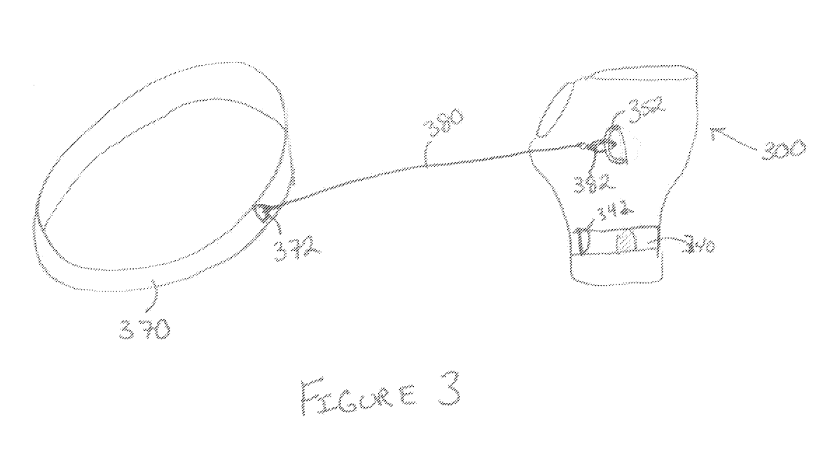

FIG. 3 depicts a perspective view of correctional brace for shoulder remediation having an anchor brace in accordance with an alternative embodiment of the present invention. In some embodiments, rather than an anchor strap passing around an anchor base, such as the individual's thigh, the correction brace 300 may comprise an anchor brace 370, in addition to the wrist guard. The anchor brace 370 may comprise any band-like apparatus for wrapping around an anchor base. In many embodiments, the anchor brace 370 may comprise a strap having hook and loop fasteners thereon. In another embodiment, the anchor brace 370 may comprise a band, such as a headband or wristband type, for fitting around an anchor base. In another alternative embodiment, the anchor brace 370 may be a portion of an article of clothing, for example, pajama pants.

The anchor brace 370 may comprise an anchor connection means 372 for connecting with the anchor strap 380. In many embodiments, the anchor connection means 372 may comprise the same structure as the anchor support 352. For example a D-ring assembly.

In the embodiment shown, the anchor strap 380 may need only be sufficient length for connecting the outer surface of the anchor brace to the top surface of the wrist guard (e.g., between about 2 inches to about 4 inches), although this may be any length suitable, depending on the nature of the anchor base utilized therewith. Generally, the anchor strap 380 may have connectors at each end for mating with the anchor connection means 372 and the anchor support 352, respectively.

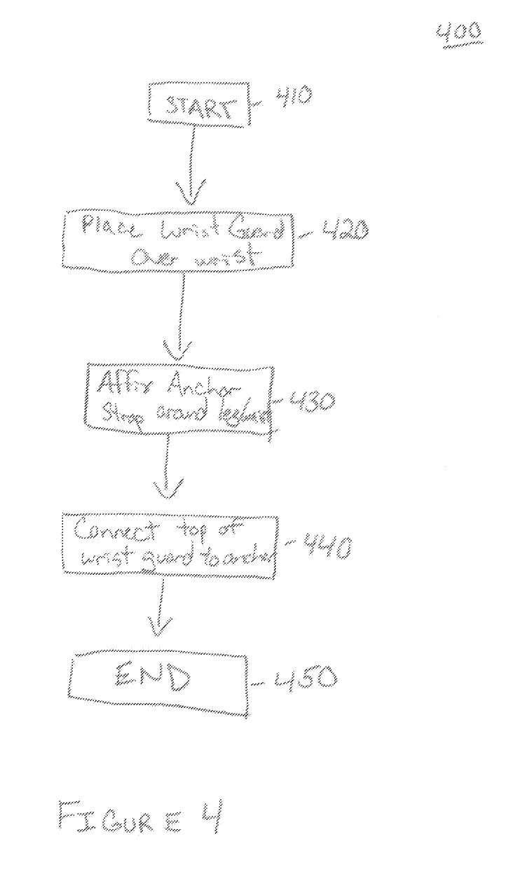

FIG. 4 depicts a flowchart of a method of utilizing a correctional brace for shoulder remediation in accordance with one embodiment of the present invention. The method 400 generally begins at step 410. At step 420, an individual will put the wrist guard securely around the individual's hand. As discussed above, the wrist guard will generally have an anchor support on a top outer surface thereof. In many embodiments, the individual will put his or her hand through the general opening of the wrist guard, and if provided, a thumb will go through the thumb hole and the fingers through the fingers hole. Optionally, the individual may secure the wrist guard with a strap assembly to ensure it is secure around his or her wrist.

At step 430, the individual will affix the anchor brace around his or her leg. Generally, if provided as a strap, the individual will place the anchor brace at a location on his or her thigh proximate the location where the individual's wrist normally lays in ordinary rest position. The anchor brace may generally be secured through one or more fastening and/or tightening means.

At step 440, the individual will connect the anchor support on the top of the wrist guard to the anchor brace via the anchor strap. In many embodiments, the anchor strap is only sufficiently long to enable the wrist guard to lay palm anterior, palm up next to the individual's thigh in a rested position. In some embodiments, where necessary, the individual may be able to tighten the anchor strap, using traditional tightening mechanisms, to ensure proper fit. Once connected, the individual's shoulder is properly positioned to ensure optimal remediation and low risk of further impingement.

The method 400 ends at step 450.

It should be emphasized that the above-described embodiments of the present invention are merely possible examples of implementations, merely set forth for a clear understanding of the principles of the invention. Many variations and modifications may be made to the above-described embodiment(s) of the invention without departing substantially from the spirit and principles of the invention. For example, an element disclosed by one embodiment of the present invention may be included in any other disclosed embodiment, where suitable. All such modifications and variations are intended to be included herein within the scope of this disclosure and the present invention and protected by the following claims.

What is claimed is:

1. A correctional brace for shoulder remediation comprising:
a wrist guard for securely fitting around an individual's hand, the wrist guard having an anchor support on a top outer surface thereof; and
an anchor strap for anchoring the wrist guard in a substantially fixed palm anterior position, the strap being connected to the anchor support, and having sufficient length to pass about an anchor base;
wherein the correctional brace is capable of decompressing the individual's shoulder and subacromial space therein.

2. The correctional brace for shoulder remediation of claim 1, wherein the wrist guard further comprises a thumb hole and a fingers hole.

3. The correctional brace for shoulder remediation of claim 1, wherein the wrist guard further comprises a strap for retaining the wrist guard on the individual's hand.

4. The correctional brace for shoulder remediation of claim 3, wherein the strap comprises a mating set of hook and loop fasteners thereon.

5. The correctional brace for shoulder remediation of claim 1, wherein the anchor support comprises a D-ring assembly on the top surface of the wrist guard.

6. The correctional brace for shoulder remediation of claim 1, wherein the anchor strap for anchoring the wrist guard comprises a mating set of hook and loop fasteners thereon.

7. The correctional brace for shoulder remediation of claim 1, further comprising:
an anchor brace for fitting around the anchor base.

8. The correction brace for shoulder remediation of claim 7, wherein the anchor brace comprises an anchor connection means for attaching to the anchor strap.

9. A correctional brace for shoulder remediation comprising:
a wrist guard for securely fitting around an individual's hand, the wrist guard having an anchor support on a top outer surface thereof;
an anchor brace for fitting around an anchor base, the anchor brace having an anchor connection means on an outer surface thereof; and
an anchor strap for anchoring the wrist guard in a substantially fixed palm anterior position, the strap connecting to the anchor support and the anchor connection means;
wherein the correctional brace is capable of decompressing the individual's shoulder and subacromial space therein.

10. The correctional brace for shoulder remediation of claim 9, wherein the wrist guard further comprises a thumb hole and a fingers hole.

11. The correctional brace for shoulder remediation of claim 9, wherein the wrist guard further comprises a strap for retaining the wrist guard on the individual's hand.

12. The correctional brace for shoulder remediation of claim 11, wherein the strap comprises a mating set of hook and loop fasteners thereon.

13. The correctional brace for shoulder remediation of claim 9, wherein the anchor support comprises a D-ring assembly on the top surface of the wrist guard.

14. The correctional brace for shoulder remediation of claim 9, wherein the anchor strap for anchoring the wrist guard comprises a mating set of hook and loop fasteners thereon.

15. A method for remediating an individual's shoulder impingement comprising:
providing a correctional brace comprising:
a wrist guard for securely fitting around an individual's hand, the wrist guard having an anchor support on a top outer surface thereof;
an anchor brace for fitting around the individual's thigh, the anchor brace having an anchor connection means on an outer surface thereof; and
an anchor strap for anchoring the wrist guard in a substantially fixed palm anterior position, the strap connecting to the anchor support and the anchor connection means;
affixing the wrist guard about the individual's wrist, and securing the wrist guard thereon;
affixing the anchor brace about an anchor base and securing the anchor brace thereon; and
connecting the anchor strap to the anchor support and the anchor connection means;
wherein the correctional brace is capable of decompressing the individual's shoulder and subacromial space therein.

16. The method of claim 15, wherein the wrist guard further comprises a thumb hole and a fingers hole.

17. The method of claim 15, wherein the wrist guard further comprises a strap for retaining the wrist guard on the individual's hand.

18. The method of claim 17, wherein the strap comprises a mating set of hook and loop fasteners thereon.

19. The method of claim 15, wherein the anchor support comprises a D-ring assembly on the top surface of the wrist guard.

20. The correctional brace for shoulder remediation of claim 15, wherein the anchor strap for anchoring the wrist guard comprises a mating set of hook and loop fasteners thereon.
